(19) United States
(12) Patent Application Publication
(10) Pub. No.: US 2017/0095606 A1
(43) Pub. Date: Apr. 6, 2017

(54) Integrated Double Cannula for ECMO

(71) Applicant: Samsung Life Public Welfare Foundation, Seoul (KR)

(72) Inventor: Yang Hyun Cho, Seoul (KR)

(73) Assignee: Samsung Life Public Welfare Foundation, Seoul (KR)

(21) Appl. No.: 15/315,852

(22) PCT Filed: Jun. 26, 2015
(86) PCT No.: PCT/KR2015/006550

§ 371 Date: Dec. 2, 2016

(30) Foreign Application Priority Data
Jun. 26, 2014 (KR) 20-2014-0004832
Jun. 26, 2015 (KR) 10-2015-0090945

(51) Int. Cl. A61M 1/36; A61M 25/00; A61M 25/01; A61M 1/16

(52) U.S. Cl. CPC A61M 1/3659; A61M 1/16; A61M 25/003; A61M 25/0108; A61M 2025/0031; A61M 2025/0037

(57) ABSTRACT

The present invention relates to an integrated double cannula for ECMO. The integrated double cannula is configured to be inserted into a blood vessel through skin for ECMO. The integrated double cannula includes a cannula body in which a blood introduction part and a blood supply part are integrally formed inside a single pipe with a partition wall being therebetween, wherein an end of the cannula body is opened through an end hole, another end of the cannula body is opened through an outlet and an inlet arranged side by side and connected respectively to the blood introduction part and the blood supply part, and one or more side holes and one or more discharge holes connected respectively to the blood introduction part and the blood supply part are formed in a lateral portion of the cannula body.

### TECHNICAL FIELD

The present invention relates to an integrated double cannula for ECMO, and more particularly, to an integrated double cannula for ECMO configured to be inserted into a blood vessel, the integrated double cannula including a blood introduction part and a blood supply part integrally formed with a partition wall being therebetween, and a plurality of through-holes formed in a lateral portion of the integrated double cannula to increase the rate of blood flowing into an ECMO system.

### BACKGROUND ART

In general, extracorporeal membrane oxygenation (ECMO) refers to emergency cardiopulmonary bypass treatment for assisting bodily functions of patients such as the cardiopulmonary function of end-stage cardiac failure or respiratory failure patients.

ECMO is frequently used as emergency treatment for patients in cardiac arrest or failure due to coronary artery disease.

ECMO for providing temporary cardiopulmonary support to patients is generally of two types. One type is veno-arterial ECMO used to assist both the cardiac and pulmonary functions of patients, and the other type is veno-venous ECMO used to assist only the pulmonary function of patients. Particularly, in the veno-venous ECMO, cannulas connected to an end of an ECMO system are respectively inserted into a jugular vein of the neck and a femoral vein of a thigh. In most cases, blood drained through the cannulas connected to the femoral vein is circulated in the ECMO system and is then returned to the heart through the jugular vein. A cannula insertable into a jugular vein and enabling both the draining and returning of blood has been developed to substitute for the use of two cannulas at a time and thus to improve surgical procedures and patient convenience.

The above-mentioned cannula of the related art is made by connecting two cannulas side by side to have a diameter corresponding to the diameter of a jugular vein. However, the cannula of the related art is limited by the diameter of a jugular vein, that is, a blood vessel, and thus has a limited diameter, and the length of the cannula is also short. Therefore, the blood flow rate of the cannula is markedly limited.

### DETAILED DESCRIPTION OF THE INVENTION

### Technical Problem

The patent document discloses a system for mixing a fluid into a bloodstream of a body, the system including: a drainage cannula adapted to be inserted through both a superior vena cava of a heart of the body and an inferior vena cava of the heart; the drainage cannula having a first opening to receive blood from a superior vena cava blood flow and a second opening to receive blood from an inferior vena cava blood flow; an infusion tube adapted to be inserted into a bloodstream downstream of both the superior vena cava and inferior vena cava; the drainage cannula and the infusion tube being coupled as a single cannula assembly with at least two lumens, one lumen being the drainage cannula and the other lumen being the infusion tube; a septum coupled between the lumens to fluidicly separate the lumens along at least a portion of a length of each lumen and adapted to flex between the two lumens to vary a cross-sectional flow area of at least one of the lumens; a pump coupled to the drainage cannula and the infusion tube; a mixing unit coupled to the pump and adapted to mix the fluid into the bloodstream of the body; and an atraumatic introducer coupled to the cannula assembly and sized larger than a cross-sectional dimension of at least one of the lumens and adapted to pass through such a lumen and cause the septum to flex toward another lumen to allow passage of the introducer.

However, the cannula is limited to the diameter of a jugular vein, that is, a blood vessel, and has a limited thickness and length. Thus, the blood flow rate of the cannula is markedly limited.

In addition, since an introduction part and an discharge port of the cannula through which blood is introduced and discharged are adjacent to each other, the cannula occupies a large space. Furthermore, since the cannula has only one side hole through which blood is discharged to an ECMO system, the amount of blood supplied to the EMC, that is, the flow rate of blood, is small, and thus blood is not smoothly oxygenated. As a result, a larger amount of blood has to be recirculated, and thus the efficiency of ECMO is low.

In addition, since the cannula of the related art is short, blood may not be smoothly drained and returned through the cannula inserted in a jugular vein of the neck of a patient if the patient moves his/her neck or face. Furthermore, the patient may be uncomfortable because of insertion of the cannula having a relatively large size.

To solve the above-mentioned problems, the present invention provides an integrated double cannula for ECMO. The integrated double cannula is configured to be inserted into a femoral vein and includes two passages therein for introducing and supplying blood, wherein the two passages are defined by a partition wall, at least one through-hole is formed in a lateral portion of the cannula to increase the flow rate of blood flowing to an ECMO system, and even though a large amount of blood is introduced into the ECMO system, the large amount of blood is handled to increase the efficiency of surgical treatment.

### Technical Solution

To achieve the above-mentioned objects, the present invention provides an integrated double cannula configured to be inserted into a blood vessel for ECMO, the integrated double cannula including a cannula body in which a blood introduction part and a blood supply part are integrally formed inside a single pipe with a partition wall being therebetween, wherein an end of the cannula body is opened through an end hole, another end of the cannula body is opened through an outlet and an inlet arranged side by side and connected respectively to the blood introduction part and the blood supply part, and one or more side holes and one or more discharge holes connected respectively to the blood introduction part and the blood supply part are formed in a lateral portion of the cannula body.

Preferably, the integrated double cannula further includes a ring-shaped metallic marker around a rear end region of the discharge holes so that an end portion of the cannula body is perceived in a radiographic image.

Preferably, a front end portion of the blood supply part has a slope inclined toward the outlet such that blood introduced through the end hole flows smoothly to an ECMO system.

Preferably, the partition wall is provided in the cannula body to separate the blood introduction part and the blood supply part, and the partition wall includes a flexible material.

Preferably, the flexible material of the partition wall is a film-shaped synthetic resin material.

Preferably, the integrated double cannula further includes a protective flange configured to be fitted into an insertion hole formed in skin to receive the cannula body.

### Advantageous Effects of the Invention

The integrated double cannula for ECMO of the present invention has the following effects. First, owing to the one or more side holes, blood may be introduced into the ECMO system at a high rate, and even though a large amount of blood is introduced into the ECMO system, the blood may be handled, thereby increasing the efficiency of surgical treatment.

Secondly, owing to the one or more side holes, the flow rate of blood increases, and a blood recirculation phenomenon reduces markedly unlike in the related art, thereby increasing efficiency.

Thirdly, since the partition wall is formed of a film-shaped synthetic resin material, the shape of the partition wall may be freely varied to increase the space of the blood introduction part or the blood supply part, thereby increasing the flow rate of blood.

Fourthly, since the length of the cannula body is about 70 cm and greater than the length of conventional cannulas, even though the cannula body is somewhat moved inward or outward during surgical treatment, the surgical treatment may be safely performed without problems.

Fifthly, since the cannula body is configured to be inserted into a femoral vein, the integrated double cannula may be used for patients whose jugular veins are difficult to access, such as patients having surgical experience on their neck, jugular vein thrombosis, or a central vein catheter in their jugular vein, or patients in a very emergent situation such as a cardiopulmonary resuscitation situation.

Sixthly, during ECMO, the integrated double cannula of the present invention causes fewer complications such as heart injury than cannulas configured to be inserted into a cervical portion, and thus ECMO may be safely performed.

Seventhly, the protective flange may be fitted onto skin to receive the cannula while preventing the introduction of contaminants into a blood vessel.

### DESCRIPTION OF THE DRAWINGS

FIG. 1 is a view illustrating a structure oldie related art.

FIG. 2 is a perspective view illustrating an integrated double cannula for ECMO according to the present invention.

FIG. 3 is a partially enlarged cross-sectional view illustrating a main portion of the integrated double cannula illustrated in FIG. 2.

FIG. 4 is a cross-sectional view illustrating a state in which the integrated double cannula of the present invention is used together with a protective flange.

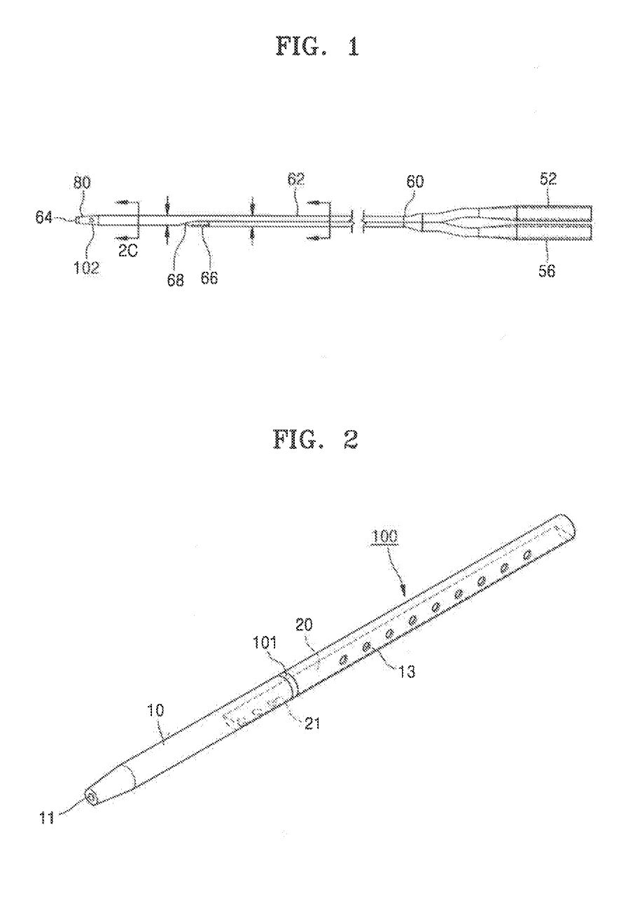

### BEST MODE

Preferred embodiments of the present invention will now be described with reference to the accompanying drawings to help understanding of the present invention. The embodiments of the present invention may be variously modified, and thus it should not be construed that the scope of the present invention is limited to the embodiments described below in detail. The embodiments are provided to help those of ordinary skill in the art to fully understand the present invention. In the drawings, the shape of elements may be exaggerated for clear description. In addition, like reference numerals may denote like elements throughout the drawings. Furthermore, detailed descriptions related to well-known functions or configurations may be omitted in order not to unnecessarily obscure subject matters of the present invention.

Hereinafter, an integrated double cannula for ECMO will be described in detail according to preferred embodiments of the present invention with reference to the accompanying drawings.

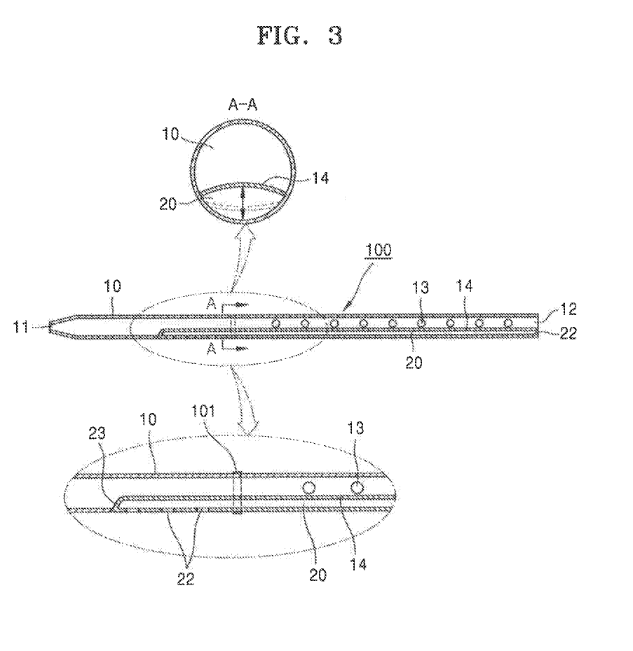

FIG. 2 is a perspective view illustrating the integrated double cannula for ECMO of the present invention, and FIG. 3 illustrates a cross section and an enlarged cross section of a main portion of the integrated double cannula illustrated in FIG. 2.

Referring to FIGS. 2 and 3, the integrated double cannula for ECMO of the present invention is shaped like a cylindrical pipe and includes a cannula body 100. The cannula body 100 is divided by an inner partition wall 14 into a blood introduction part 10 providing a blood path toward an ECMO system and a blood supply part 20 through which blood introduced into the ECMO system via the blood introduction part 10 is returned to a blood vessel after being oxygenated.

The blood introduction part 10 and the blood supply part 20 will now be described in detail. The blood introduction part 10 has a pipe shape, and an end hole 11 is formed in an end portion of the blood introduction part 10 to introduce blood. In addition, an outlet 12 is formed in another end portion of the blood introduction part 10 to discharge blood introduced through the end hole 11, and a plurality of side holes 13 are formed in a lateral portion of the blood introduction part 10.

The blood supply part 20 is separated from the blood introduction part 10 by the partition wall 14 formed on a side of the blood introduction part 10. A front portion of the blood supply part 20 is closed, and the blood supply part 20 includes a plurality of discharge holes 21 in a lateral portion thereof and an inlet 22 in a rear end portion thereof. Therefore, blood introduced into the blood introduction part 10 may be supplied back to a blood vessel after the blood passes through the ECMO system and the inlet 22. The partition wall 14 may be formed of a flexible material such as a synthetic resin material having a film shape. The synthetic resin material may be a plastic film such as a polyethylene film, an LDPE film, or an LLDPE film. In general, the partition wall 14 may be formed of a polyethylene film. Alternatively, the partition wall 14 may be formed of a recently developed biodegradable plastic film containing a biodegradable polymer. Therefore, as shown in the enlarged cross-section view of FIG. 3, since the partition wall 14 is formed of a flexible material such as a synthetic resin film, when blood is introduced into the blood introduction part 10, the partition wan 14 may be pushed toward a pipe inner wall, and thus an inner space of the blood introduction part 10 may be increased. Thus, the inflow rate of blood may be increased, In addition, when blood is supplied to the blood supply part 20, the partition wall 14 may be pushed inward and expanded, and thus an inner space of the blood supply part 20 may be increased. Therefore, the supply rate of blood may be increased.

In addition, a slope 23 inclined toward the outlet 12 is formed on a front end portion of the blood supply part 20, that is, a front end portion of the partition wall 14. Thus, blood introduced through the end hole 11 may be smoothly introduced into the ECMO system.

The cannula body 100 may be inserted into a femoral vein to supply oxygenated blood toward the tricuspid valve of the heart, and the blood introduction part 10 and the blood supply part 20 are integrally formed in a single pipe.

Therefore, the outlet 12 and the inlet 22 are arranged in parallel up and down the same site and the blood supply part 20 is located on a side of the blood introduction part 10,

Preferably, the cannula body 100 may have a length of 65 cm to 75 cm, and most preferably 70 cm. The length from the outlet 12 and the inlet 22 to the last one of the side holes 13 is about 40 cm, and the length from the last side hole 13 to the end hole H is about 30 cm.

The cannula body 100 further includes a metallic marker 101 formed around a rear end region of the discharge holes 21 so that an end portion of the cannula body 100 may be easily perceived in a radiographic image. Therefore, when the cannula body 100 is inserted into a femoral vein, the insertion depth of the cannula body 100 may be easily checked using ultrasonic waves or X-rays.

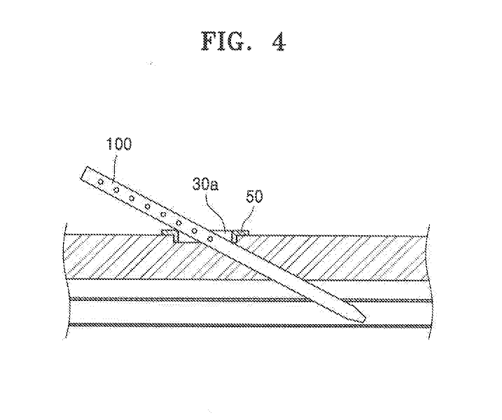

FIG. 4 is a cross-sectional view illustrating a state in which the integrated double cannula for ECMO is used together with a protective flange according to the present invention.

As illustrated in FIG. 4, preferably, the protective flange 50 is fitted in an insertion hole 30a formed in skin 30, and then the cannula body 100 is inserted into a blood vessel 40 through the insertion hole 30a of the skin 30. Thus, since a gap formed in the insertion hole 30a between the cannula body 100 and the skin 30 is blocked by the protective flange 50, foreign substances or contaminants may not be introduced into the blood vessel 40 through the skin 30, and thus the blood vessel 40 may not be contaminated.

### MODE OF THE INVENTION

Hereinafter, an explanation will be given of how the integrated double cannula having the above-described structure for ECMO is used according to the present invention.

First, the cannula body 100 is inserted into a femoral vein of a patient requiring ECMO.

Thereafter, as an ECMO system is operated, blood is introduced into the blood introduction part 10 through the end hole 11 and the side holes 13. Then, the inner space of the blood introduction part 10 is enlarged, and the blood is supplied to the ECMO system from the blood introduction part 10 through the outlet 12. In the ECMO system, the blood is oxygenated.

Thereafter, the oxygenated blood is introduced into the blood supply part 20 through the inlet 22, and then the inner space of the blood supply part 20 is enlarged. The oxygenated blood is supplied to the femoral vein from the blood supply part 20 through the discharge holes 21.

At this time, since the number of the side holes 13 is three or more, blood may be introduced into the ECMO system at a high rate. Although a large amount of blood is introduced into the ECMO system, the large amount of blood may be handled, and thus the efficiency of surgical treatment may be increased.

In addition, since the length of the cannula body 100 is about 70 cm, even though the cannula body 100 is somewhat moved inward or outward during surgical treatment, the surgical treatment may be safely performed without problems.

Furthermore, since the cannula body 100 is inserted into a femoral vein instead of inserting a conventional short cannula into a jugular vein, blood may be smoothly oxygenated without being largely affected by the movement of the neck and face of a patient,

While the integrated double cannula for ECMO has been described according to embodiments of the present invention, the embodiments should be construed in a descriptive sense only, and it will be apparent to those skilled in the art that various changes and other embodiments may be made therefrom. Thus, it will be understood that the present invention is not limited to the above detailed description. Therefore, the scope and spirit of the present invention should be defined by the claims of the present utility model application. In addition, it should be understood that the present invention covers all modifications, equivalents, and replacements within the spirit and scope of the present invention defined by the claims,

### INDUSTRIAL APPLICABILITY

According to the integrated double cannula for ECMO of the present invention, blood may be supplied to an ECMO system at a high rate, and even though a large amount of blood is introduced into the ECMO system, the large amount of blood may be handled, thereby increasing the efficiency of surgical treatment.

In addition, owing to the one or more side holes, the flow rate of blood increases, and a blood recirculation phenomenon reduces markedly. Thus, the integrated double cannula may be used to efficiently perform ECMO.

In addition, since the partition wall is formed of a film-shaped synthetic resin material, the shape of the partition wall may be freely varied to increase the space of the blood introduction part or the blood supply part. Thus, the integrated double cannula may have a high blood flow rate and thus may be usefully used for ECMO,

In addition, since the length of the cannula body is about 70 cm and greater than the length of conventional cannulas, even though the cannula body is somewhat moved inward or outward during surgical treatment, the surgical treatment may be performed without problems, Thus, the integrated double cannula of the present invention may be safely used for ECMO, In addition, since the cannula body is configured to be inserted into a femoral vein, the integrated double cannula may be used for patients whose jugular veins are difficult to access, such as patients having surgical experience on their neck, jugular vein thrombosis, or a central vein catheter in their jugular vein, or for patients in a very emergent situation such as a cardiopulmonary resuscitation situation. Thus, the integrated double cannula may be usefully used for ECMO.

In addition, during ECMO, the integrated double cannula causes fewer complications such as heart injury than cannulas configured to be inserted into a cervical portion. Thus, the integrated double cannula may be safely used for ECMO.

In addition, the protective flange may be fitted onto skin to receive the integrated double cannula while preventing the introduction of contaminants into a blood vessel.

1. A integrated double cannula configured to be inserted into a blood vessel for ECMO, the integrated double cannula comprising a cannula body in which a blood introduction part and a blood supply part are integrally formed inside a single pipe with a partition wall being therebetween, wherein an end of the cannula body is opened through an end hole, another end of the cannula body is opened through an outlet and an inlet arranged side by side and connected respectively to the blood introduction part and the blood supply part, and one or more side holes and one or more discharge holes connected respectively to the blood introduction part and the blood supply part are formed in a lateral portion of the cannula body.

2. The integrated double cannula of claim 1, further comprising a ring-shaped metallic marker around a rear end region of the discharge holes so that an end portion of the cannula body is perceived in a radiographic image.

3. The integrated double cannula of claim 1, wherein a front end portion of the blood supply part has a slope inclined toward the outlet such that blood introduced through the end hole flows smoothly to an ECMO system.

4. The integrated double cannula of claim 1, wherein the partition wall is provided in the cannula body to separate the blood introduction part and the blood supply part, and the partition wall comprises a flexible material.

5. The integrated double cannula of claim 4, wherein the flexible material of the partition wall is a film-shaped synthetic resin material.

6. The integrated double cannula of claim 1, further comprising a protective flange configured to be fitted into an insertion hole formed in skin to receive the cannula body.
